(19) United States
(12) Patent Application Publication
(10) Pub. No.: US 2026/0306433 A1
(43) Pub. Date: Oct. 8, 2026

(54) HIERARCHICAL FATTY ACID ARCHITECTURE AND MAMMALIAN LIPID METABOLIC SYSTEM FOR NUTRITIONAL COMPOSITIONS AND METHODS OF USE

(71) Applicant: Goldie Formula Inc., Miami, FL (US)

(72) Inventors: Savannah Capasso, Miami, FL (US); Daniel J. Schmitz, Westerville, OH (US)

(21) Appl. No.: 19/641,207

(22) Filed: Apr. 7, 2026

Related U.S. Application Data
(60) Provisional application No. 63/785,120, filed on Apr. 8, 2025.

(51) Int. Cl. A61K 31/201; A23C 9/152; A23C 9/158; A23L 33/00; A61K 31/20; A61K 35/20; A61K 36/54; A61K 36/63

(52) U.S. Cl. CPC A61K 31/201; A23C 9/1526; A23C 9/1528; A23C 9/158; A23L 33/40; A61K 31/20; A61K 35/20; A61K 36/54; A61K 36/63

(57) ABSTRACT

Provided herein are compositions and methods for Hierarchical Fatty Acid Architecture and Mammalian Lipid Metabolic System for Nutritional Compositions.

### CROSS-REFERENCE TO RELATED APPLICATIONS

The present application claims priority to U.S. provisional application Ser. No. 63/785,120, filed Apr. 8, 2025, herein incorporated by reference in its entirety.

### BACKGROUND

The invention generally relates lipid systems for formulated nutritional compositions.

Commercially formulated nutritional products across infant, pediatric, and adult categories typically rely on industrial seed oils and tropical oils as primary lipid sources.

These conventional lipid systems frequently exhibit elevated polyunsaturated fatty acid concentrations, high linoleic acid levels, lauric-acid dominant saturated fat profiles, and limited monounsaturated fatty acid dominance. In addition, refined vegetable oil systems lack the biological lipid membrane structures naturally present in mammalian milk.

High polyunsaturated fatty acid exposure may increase susceptibility to lipid peroxidation due to the presence of multiple double bonds that are vulnerable to oxidative degradation.

While certain modern formulations include dairy fat or isolated milk fat globule membrane fractions, these approaches do not establish a coherent lipid architecture designed to replicate biological fatty acid hierarchy and oxidative stability observed in mammalian milk systems.

Accordingly, there remains a need for nutritional lipid systems that recreate aspects of mammalian milk lipid architecture through coordinated fatty acid class hierarchy, controlled PUFA exposure, and incorporation of mammalian lipid membrane structures.

Infant formula is strictly regulated to ensure 1) it contains all the essential basic nutrients found in breast milk needed for growth and 2) it is free from any harmful contaminants. However, there are no explicit requirements pertaining to metabolic health or any recognition of the implications processed ingredients may have on this. Mitochondrial dysfunction is implicated in obesity and other chronic diseases associated with metabolic syndrome.

There is an alarming increase in infant and child obesity rates. It is difficult to explain infant obesity without considering metabolic health. Human milk is rich in biological and natural antioxidants (from fruits and vegetables in the mom's diet). Breast feeding rates have fallen in the last 50 years while obesity has risen. Infant formula ingredients are highly processed which results in lower intrinsic antioxidant content, as well as containing higher levels of PUFAs (polyunsaturated fatty acids). Conventional infant formulas commonly derive a substantial portion of total fat from industrially refined seed oils that are relatively high in polyunsaturated fatty acids (PUFAs), particularly linoleic acid. While PUFAs are essential nutrients within defined ranges, excessive PUFA exposure increases susceptibility to lipid peroxidation due to the inherent chemical instability of multiple double bonds. This elevated oxidative burden can lead to increased formation of reactive lipid oxidation products during processing, storage, and digestion. In the context of infancy—characterized by high metabolic rate and active mitochondrial function—excessive PUFA-driven oxidative stress may place additional strain on cellular antioxidant systems and mitochondrial membranes. Accordingly, there is a need for infant formula lipid systems that meet essential fatty acid requirements while reducing unnecessary PUFA-associated oxidation.

There has been an alarming rise in obesity for infants and children over the last 50 years. The US obesity rates for children aged 2-19 years in the 1970s was about 5%. By 2018, this rate had increased to around 18.5%. (Source: US CDC). Over this same period, the rates of overweight and obesity among infants and toddlers have also increased. While statistics are harder to compile for this younger age group, studies indicate a growing prevalence coinciding with the overall rise in childhood obesity. Early obesity in this age group is concerning due to its association with longer term health risks including Type 2 diabetes, cardiovascular disease, psychological issues, and cancer, all manifestations of Metabolic Syndrome or Mitochondrial Dysfunction. These statistics also coincide with generally lower breastfeeding rates during this same period. Breastfeeding is linked to a lower risk of obesity.

Factors related to these increased obesity rates in formula fed infants is generally attributed to higher caloric intake. All infant formulas for the last 50 years have been relying on industrial seed oils which are known to be lower in antioxidants and higher in PUFAs compared to olive and avocado oils. Human milk is high in biological antioxidants and phytochemical antioxidants that pass into breast milk from the plants in a mom's diet, including fruits and vegetables. Infant metabolism is 3× the rate of an adult and is at its highest point throughout life. A byproduct of cellular metabolism, i.e. mitochondrial function, are Reactive Oxygen Species (ROS). These are free radicals that if are not quenched by biological or other antioxidants, can harm and damage the mitochondria-a cell's engine. A continued assault on the mitochondria without antioxidant protection can damage the mitochondria and ultimately result in mitochondrial dysfunction which generally manifests as metabolic syndrome and its sequelae of obesity, type 2 diabetes, cardiovascular disease, and cancer. Breast milk is designed to protect the infants with a 3× metabolic rate. Infant formula using industrial seed oils is poorly equipped to do this because of low intrinsic antioxidants and the oxidative burden contributed by the higher levels of PUFAs. Polyunsaturated fatty acids (PUFAs) contain multiple carbon-carbon double bonds that confer essential biological functions but also render these lipids inherently more susceptible to oxidative degradation. Each additional double bond increases the probability of free-radical attack, leading to the formation of propagating cascading lipid peroxides and secondary reactive aldehydes during processing, storage, and post-prandial metabolism.

Mitochondria are both a primary source and a primary target of reactive oxygen species (ROS). During oxidative phosphorylation, electron leakage from the respiratory chain generates ROS as a normal byproduct of cellular metabolism. Lipid peroxidation products derived from PUFA-rich lipid substrates can amplify this oxidative burden by propagating radical chain reactions and interacting with mitochondrial membranes, which are themselves lipid-dense and essential to respiratory efficiency.

Excessive PUFA-derived oxidative stress may disrupt mitochondrial membrane integrity, alter membrane fluidity, and impair electron transport efficiency. Such effects can increase mitochondrial inefficiency, elevate compensatory ROS generation, and reduce metabolic flexibility. Over time, this self-reinforcing cycle of oxidative stress and mitochondrial strain may contribute to metabolic dysregulation characterized by altered energy utilization, impaired lipid handling, and inflammatory signaling pathways commonly associated with metabolic syndrome.

In infancy, mitochondrial activity and metabolic rate are markedly elevated relative to later life stages, increasing sensitivity to oxidative imbalances. Accordingly, infant nutrition systems that deliver essential fatty acids while minimizing unnecessary PUFA-associated oxidative stress may provide functional advantages in supporting mitochondrial resilience and long-term metabolic health.

It's our hypothesis that this is a significant contributor to the alarming increase in infant obesity. The arguments usually given for obesity including 1) hyper palatability 2) calorically dense foods, 3) lack of physical activity and sleep do not generally pertain to an infant. A better explanation is the assault on the mitochondrial function from a lack of protective antioxidants or elevated ROS assault from oxidized PUFAs. The present invention attempts to solve these problems, as well as others.

### SUMMARY OF THE INVENTION

Provided herein are compositions and methods for Hierarchical Fatty Acid Architecture and Mammalian Lipid Metabolic System for Nutritional Compositions.

The methods and compositions are set forth in part in the description which follows, and in part will be obvious from the description, or can be learned by practice of the methods and compositions. The advantages of the methods and compositions will be realized and attained by means of the elements and combinations particularly pointed out in the appended claims. It is to be understood that both the foregoing general description and the following detailed description are exemplary and explanatory only and are not restrictive of the methods and compositions, as claimed.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53 (c) EPC and Rule 28 (b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

### BRIEF DESCRIPTION OF THE DRAWINGS

In the accompanying figures, like elements are identified by like reference numerals among the several preferred embodiments of the present invention.

FIG. 1 is a table of formulation that shows the risk of receiving all oils with fatty acids at the low end of the specification range for linoleic acid.

FIG. 2 is a table of a formulation that shows the fatty acid profile achievable by increasing the minimum spec value for linoleic acid to 8.5% for both Avocado Oil and Olive oil (from 5% and 3.5%, respectively).

FIG. 3 is a table of a formulation that shows the linoleic acid profile achievable by using the mid-point spec value for all oils.

FIG. 4 is a graph showing Oxidative Stability Index (OSI) for the oil blend of the present invention (designated as FN518090 C) having a higher comparative oxidative stability at 15.31 OSI (h @ 110C) and 63.66 OSI (h @ 90C) than both commercial blends (designated as FN518090 A and FN518090 B), where both methods used (standard 110° C., as well as the 90° C.).

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

Embodiments of the invention will now be described with reference to the Figures, wherein like numerals reflect like elements throughout. The terminology used in the description presented herein is not intended to be interpreted in any limited or restrictive way, simply because it is being utilized in conjunction with detailed description of certain specific embodiments of the invention. Furthermore, embodiments of the invention may include several novel features, no single one of which is solely responsible for its desirable attributes or which is essential to practicing the invention described herein.

The use of the terms “a” and “an” and “the” and similar referents in the context of describing the invention are to be construed to cover both the singular and the plural, unless otherwise indicated herein or clearly contradicted by context. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

Recitation of ranges of values herein are merely intended to serve as a shorthand method of referring individually to each separate value falling within the range, unless otherwise indicated herein, and each separate value is incorporated into the specification as if it were individually recited herein. The word “about,” when accompanying a numerical value, is to be construed as indicating a deviation of up to and inclusive of 10% from the stated numerical value. The use of any and all examples, or exemplary language (“e.g.” or “such as”) provided herein, is intended merely to better illuminate the invention and does not pose a limitation on the scope of the invention unless otherwise claimed. No language in the specification should be construed as indicating any nonclaimed element as essential to the practice of the invention.

References to “one embodiment,” “an embodiment,” “example embodiment,” “various embodiments,” etc., may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

### Definitions

The term “infant” means a child under the age of 12 months.

The expression “young child” means a child aged between one and three years, also called toddler.

An “infant or young child born by C-section” means an infant or young child who was delivered by caesarean. It means that the infant or young child was not vaginally delivered.

An “infant or young child vaginally born” means an infant or young child who was vaginally delivered and not delivered by caesarean.

A “preterm” or “premature” means an infant or young child who was not born at term. Generally, it refers to an infant or young child born prior 36 weeks of gestation.

In some embodiments, the “infant formula” is for a preterm or full-term infant. As used herein, a “preterm” birth refers to an alive birth before 37 weeks of pregnancy are completed. There are sub-categories of preterm birth, based on gestational age: extremely preterm (less than 28 weeks) very preterm (28 to 32 weeks) moderate to late preterm (32 to 37 weeks). In the context of this disclosure, a “term” birth refers to an alive birth anytime between 37 weeks and 42 weeks of pregnancy, and a “full term” birth is an alive birth during 39 weeks and 40 weeks of pregnancy.

The term “synergistic” as used herein is refers to the phenomenon wherein the cumulative pharmacological effect of two or more ingredients when used in combination is higher than the sum of the effect of each of them tested individually. The term “potentiating” as used herein refers to the phenomenon where the efficacy of an active ingredient is significantly enhanced when it is combined with a second ingredient, wherein said second ingredient itself does not demonstrate any efficacy in the same pharmacological test. In some cases of potentiation, not only is said second ingredient devoid of the pharmacological effect being measured, it may even cause an opposite effect, when assayed alone. An example of such a case would be as follows: ingredient A is anti-inflammatory; ingredient B is pro-inflammatory; when A and B are combined, said combination produces an anti-inflammatory effect that is greater than seen with A alone. In the context of the present invention, potentiation is regarded as a special case of synergism. Thus, the term ‘synergism’ (or synergistic, or the like), when used to define the properties of a composition of the present invention, also includes within its range of meaning the potentiation effect described immediately hereinabove.

In the present disclosure, an “effective amount” of an agent is an amount of such an agent that is sufficient to effect beneficial or desired results or desired physiologic effect as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other agents being administered, the age, size, and species of the subject, and like factors well known in the arts of, e.g., nutritional formula or supplementation. In general, a suitable dose of an agent according to the disclosure will be that amount of the agent, which is the lowest dose effective to produce the desired effect with no or minimal side effects.

Infant Formula can be used as a sole source of nutrition from 0-12 months for both term and pre-term infants. It can also be used as sole source or complementary nutrition from 1-2 years of age.

Follow On Formula is complementary nutrition that is generally used between 1-5 years of age.

Supplement Formulation is a complementary nutrition that may be used between years 5-120 years of age.

The composition of the invention is an oral formula or formulation, and the dosage form of the oral formula is oral liquid, tablet, powder, capsule or granule. Preferred oral formulations are those which can be taken with the addition of infant formula, water, milk, or breast milk.

As used herein, “nutritional compositions” or “formulated nutritional compositions” refers to nutritional products intended for consumption.

### DESCRIPTION OF EMBODIMENTS

Generally speaking, the invention relates to lipid systems for formulated nutritional compositions or formulas including a lipid architecture with structural and metabolic characteristics of mammalian lipid systems. The lipid architecture is defined as a Hierarchical Fatty Acid Architecture (HFAA) and is characterized by coordinated fatty acid class distribution, controlled polyunsaturated fatty acid exposure, and integration of mammalian-derived lipid structures.

The architecture may comprise: Monounsaturated Fatty Acid (MUFA) class dominance; moderated saturated fatty acid contribution; constrained Polyunsaturated Fatty Acid (PUFA) exposure; restricted linoleic acid concentration; moderated lauric acid contribution; inclusion of mammalian-derived fat fractions; optional inclusion of ruminant-derived milk fat globule membrane (MFGM) components. The lipid architecture influences oxidative stability, digestion kinetics, lipid absorption dynamics, and metabolic signaling processes.

The present invention provides a nutritional composition comprising a lipid system exhibiting a Hierarchical Fatty Acid Architecture (HFAA). In certain embodiments the architecture comprises: a total fat acid content is between about 20% and about 60% of caloric value of the composition; monounsaturated fatty acids comprising between about 55% and about 75% of total fatty acids; saturated fatty acids comprising between about 15% and about 30% of total fatty acids; polyunsaturated fatty acids comprising no more than about 15% of the total fatty acid content of the composition; linoleic acid comprising between about 6% and about 12% of the total fatty acid content of the composition; lauric acid comprising less than about 6% of total fatty acids; and including at least one mammalian-derived fat source. Alternatively, the lauric acid comprising less than about 4% of total fatty acids.

In certain embodiments, the total fat is between about 15% and about 70% of caloric value of the composition; alternatively, between about 20% and about 55%, alternatively, between about 28% and about 50%. In certain embodiments, the monounsaturated fatty acids comprise between about 50% and about 80% of the total fatty acids, alternatively between about 60% and about 70%, alternatively between about 62% and about 68%. In certain embodiments, the saturated fatty acids comprise between about 10% and about 35% of the total fatty acids, alternatively between about 20% and about 28%, alternatively between about 22% and about 26%. In certain embodiments, the polyunsaturated fatty acids comprise no more than about 20% of the total fatty acid content of the composition, alternatively, no more than about 18%, alternatively, no more than about 12%, alternatively, no more than 10%. In certain embodiments, the linoleic acid comprises between about 4% and about 15% of the total fatty acid content of the composition, alternatively, between about 8% and about 14%, alternatively, between about 9% and about 11%. In certain embodiments, the lauric acid comprises no more than between about 2% and about 8% of total fatty acids, alternatively, between about 3% and about 7%, alternatively, between about 4% and about 6%.

In certain embodiments the mammalian-derived fat fraction contains milk fat globule membrane components originating from ruminant dairy fat fractions.

The resulting lipid architecture comprises: improved oxidative stability; moderated digestion kinetics; reduced lipid peroxidation susceptibility; metabolic signaling characteristics resembling mammalian milk lipid systems.

Mammalian milk represents a biologically evolved nutritional system optimized to support growth, metabolic stability, and developmental signaling during early life. Unlike many modern formulated lipid systems, mammalian milk lipids are not random mixtures of oils but exhibit a coherent structural organization that integrates fatty acid distribution, lipid membrane structures, and lipid delivery mechanisms.

### 1. Fatty Acid Class Hierarchy

Mammalian milk lipids demonstrate functional hierarchy in which monounsaturated fatty acids contribute significantly to total lipid composition while saturated fats provide structural energy density and polyunsaturated fatty acids remain moderated.

### 2. Controlled Polyunsaturated Fatty Acid Exposure

Mammalian milk typically maintains polyunsaturated fatty acids within moderate ranges rather than allowing them to dominate the lipid system. This moderated exposure may reduce oxidative vulnerability.

### 3. Biological Lipid Membrane Structures

Milk lipids are naturally organized into fat globules surrounded by the milk fat globule membrane (MFGM). These membranes contain phospholipids, sphingolipids, and other bioactive lipid structures that influence digestion and metabolic signaling.

### 4. Oxidative Stability Balance

The fatty acid distribution present in mammalian milk may contribute to balanced oxidative stability by maintaining significant monounsaturated fatty acid presence while limiting excessive polyunsaturated fatty acid exposure.

### 5. Metabolic Signaling Integration

Milk lipids participate in coordinated metabolic signaling processes affecting digestion kinetics, nutrient absorption, and satiety signaling. The HFAA system described herein applies these principles to formulated nutritional compositions.

### Hierarchical Fatty Acid Architecture

The invention is defined by a hierarchical lipid architecture comprising three coordinated fatty acid tiers comprising Monounsaturated Fatty Acids (MUFA Dominance), Moderated Saturated Fatty Acids, and Constrained Polyunsaturated Fatty Acids.

### Primary Tier—Monounsaturated Fatty Acids (MUFA Dominance)

Monounsaturated fatty acids such as oleic acid may comprise approximately between about 50% and about 75% of total fatty acids and provide oxidative stability and metabolic energy delivery. Monounsaturated fatty acids (MUFAs) are healthy, liquid-at-room-temperature fats that include oleic acid, palmitic acid, and linoleic acid.

### Secondary Tier—Moderated Saturated Fatty Acids

Saturated fatty acids may comprise between about 15% and about 30% of total fatty acids and contribute structural energy density and metabolic support. Saturated fatty acids are fat molecules without double bonds between carbon atoms, include palmitic acid (16:0), stearic acid (18:0), lauric acid (12:0), myristic acid (14:0), and among others.

### Tertiary Tier—Constrained Polyunsaturated Fatty Acids

Polyunsaturated fatty acids may be constrained to ≤15% of total fatty acids to limit oxidative susceptibility while maintaining physiological roles. Polyunsaturated fatty acids (PUFAs) including Omega-3 fatty acids and omega-6 fatty acids. Omega-3 includes primarily ALA (Alpha-Linolenic Acid), EPA (Eicosapentaenoic Acid), DHA (Docosahexaenoic Acid). Omega-6 fatty acids, primarily Linoleic Acid (LA) and Arachidonic Acid (ARA).

### Mammalian Lipid Membrane Structures

In certain embodiments the lipid system includes mammalian-derived lipid fractions containing components of the milk fat globule membrane (MFGM).

These components may include: phosphatidylcholine, phosphatidylethanolamine, sphingomyelin, gangliosides, cholesterol-associated lipid microstructures. Such structures may influence digestion kinetics, lipid absorption, and metabolic signaling processes.

### Oxidative Stability Framework

The hierarchical lipid architecture may exhibit reduced susceptibility to lipid peroxidation compared to seed-oil dominant lipid systems.

Mechanisms contributing to improved stability may include: MUFA-dominant fatty acid composition; controlled PUFA exposure; moderated linoleic acid concentration.

These characteristics may improve lipid stability during manufacturing, storage, digestion, and metabolic incorporation into cellular membranes.

The lipid architecture may be incorporated into a variety of commercial nutritional compositions including: Infant Formula Embodiment, Toddler Formula Embodiment, Pediatric Formula Embodiment, Adult Nutritional Embodiment, Medical Nutrition Embodiment.

In one embodiment, the nutritional composition including a caloric content, comprises a lipid system including a hierarchical fatty acid architecture and a total fat content: wherein the total fat content provides between about 40% and about 60% of caloric content, a monounsaturated fatty acid content comprising between about 50% and about 75% of the total fatty acid content, a polyunsaturated fatty acid content comprising no more than about 15% of the total fatty acid content, a linoleic acid content comprising between about 6% and about 12% of total fatty acid content, a lauric acid content comprising less than 6% of the total fatty acid content, and the lipid system includes at least one mammalian-derived fat source. Alternatively, the lauric acid content comprising less than 4% of the total fatty acid content

In one embodiment, the MUFA comprises between about 60% and about 70% of the total fatty acid content. In one embodiment, the PUFA comprises between about 8% and about 12% of total fatty acid content. In one embodiment, the mammalian-derived fat source comprises dairy fat. In one embodiment, the lipid system includes milk fat globule membrane components. In one embodiment, the lipid architecture modulates post-prandial metabolic signaling.

In one embodiment, the nutritional lipid system is configured to establish hierarchical fatty acid architecture that modulates metabolic signaling through coordinated fatty acid class distribution and controlled polyunsaturated fatty acid exposure. In one embodiment, the nutritional lipid architecture is designed to emulate structural characteristics of mammalian milk lipid systems through hierarchical fatty acid class distribution and inclusion of ruminant-derived lipid membrane components. In one embodiment, the nutritional composition configured to reduce incorporation of oxidatively unstable polyunsaturated fatty acids into biological membranes through constrained dietary PUFA exposure and monounsaturated fatty acid dominance.

In one embodiment, the composition is formulated as an infant formula.

In one embodiment, the formula comprises plant oils in a goat-milk or cow-milk based infant formula (IF) or follow on formula (FOF) or a supplement formula (SF). This formula comprises a formulation that promotes metabolic health and supports mitochondrial function. One hypothesis for the origin of infant obesity and its increasing rate is the inferior metabolic and mitochondrial protection in infants that are fed by infant formula. The formula comprises Oleic-Dominant, Low-PUFA Lipid Systems, with or without, higher levels of intrinsic antioxidants to provide more metabolic protection than current industrial seed oil formulasThe formula comprises an effective amount of plant oils, wherein plant oils may comprise Olive, Avocado, Flax, Chia, Walnut, Hemp oils that meet all the IFA/Codex min to max or GUL requirements for total fat energy and linoleic and α-linolenic fatty acids. Alternatively, the plant oils may comprise any other oil sources that meet all the IFA/Codex min to max or GUL requirements for total fat energy and linoleic and α-linolenic fatty acids. The IFA/Codex min to max or GUL requirements for total fat energy is between about 3.3 g/100 kcal and about 6.0 g/100 kcal for IF or between about 3.0/100 kcal and about 6.0 g/100 kcal for FOF. In one embodiment, the plant oils comprise about 5.5 g/100 kcal for IF. In one embodiment, linoleic acid comprises about 300 mg (or 2.7% of calories) for IF or FOF. In one embodiment, linoleic acid is about 360 mg. In one embodiment, alpha-linolenic acid is a minimum of 50 mg/100 kcal in both IF and FOF. In one embodiment, alpha-linolenic acid is about 60 mg. On a caloric basis, this formula provides higher levels of oleic acid (MUFA) and lower levels of linoleic (PUFA relative to formulas with industrial seed oils including Soy, Canola, Sunflower, Safflower, Corn oils. The formula also comprises higher intrinsic antioxidant levels when the extra virgin forms of Olive and/or Avocado are used relative to industrial seed oil containing formulas.

Infant formulas are important sources of key dietary components, including proteins, carbohydrates, fats, vitamins and minerals and other beneficial substances. According to certain aspects, infant formulas will have a precisely defined composition that provide protection of metabolic health and mitochondrial function in infants. For example, an infant formula can offer nutrition suitable for infants, while other formulas can provide improved outcomes as it relates to infant/toddler obesity and the sequelae of Type 2 diabetes, cardiovascular disease, psychological issues, and cancer that can result later in life.

The invention comprises a formula or formulation for infants/toddlers from 0-5 years, that comprises plant oils (Olive, Avocado) that are higher in oleic acid and lower in linoleic acid (PUFA) relative to formulas with industrial seed oils (Soy, Canola, Sunflower, Safflower, Corn). The formula also comprises higher intrinsic antioxidant levels when the extra virgin forms of Olive and/or Avocado are used relative to industrial seed oil containing formulas. As a result, it can provide better protection of metabolic health and mitochondrial function. This can support improved outcomes as it relates to infant/toddler obesity and the sequelae of Type 2 diabetes, cardiovascular disease, psychological issues, and cancer that can result later in life.

Linolenic acid is a minimum of about 50 mg/100 kcal in both IF and FOF, according to one embodiment. In one embodiment, the formula comprises linolenic acid at about 60 mg/100 kcal. In one embodiment, the formula comprises a ratio of linoleic:a linolenic between about 5:1, about 7:1, or about 15:1. In one embodiment, the formula comprises a ratio linoleic:a linolenic at about 6:1. NOTES: In one embodiment, the olive or avocado oil are interchangeable and can be used separately or together in any ratio. In one embodiment, the olive or avocado oil comprises greater than about 30% of the fat, and optionally greater than about 60%, in the formula. The formula comprises flax, chia, walnut or hemp oil alone or in any ratio in combination with the olive/avocado oils. The flax, chia, walnut or hemp oil comprise less than 10%, and optionally less than about 4%, of the total fat in the formula. Any source of Olive, Avocado, Flax, Chia, Walnut, or Hemp is viable. In one embodiment, extra virgin oil sources are preferred for maximum phytochemical content.

In one embodiment, olive oil has a range between about 3.5% and about 21% of linoleic acid and avocado oil has a range between about 5% and about 20% of linoleic acid.

The balance of the formula recipe's other ingredients including micronutrients, and optionally, DHA/AA, Nucleotides, and Prebiotics are added to ensure all current and future nutritional requirements/recommendations for infant/toddler are satisfied.

The method comprises increasing oleic acid (MUFA) and reducing linoleic acid (PUFA) and also increasing intrinsic antioxidants when virgin oils are used to protect the infant's metabolic health. This is achieved by administering a formula comprising high levels of olive and/or avocado oils, wherein the olive and/or avocado oils comprise greater than about 30%, optionally greater than about 60%, of the total fat in the formula. The method comprises meeting the IFA/CODEX requirement for α-linolenic acid and administering the formula including Flax, Chia, Walnut, or hemp oils at a level of less than about 10%, optionally less than about 4%, of the total fat in the formula. Using less than the 30% of olive and avocado oils, reduces the efficacy in promoting metabolic health in the infant. Also using less than the 30% minimum, requires either adding more than 10% of the flax, chia, walnut, or hemp oils which can adversely impact the organoleptic and oxidative stability of the formula. Substituting other allowed oils would likely increase the PUFA burden.

The best plant oils available to elevate the mitochondrial protection are the high oleic (MUFA) and low linoleic (PUFA) oils, the and avocado oils, particularly, even more so with the Extra Virgin forms.

The formula comprises dairy ingredients from goat milk or cow milk that meet the IFA/Codex minimum to maximum requirements for protein energy and quality. The protein levels may be between about 1.8 g/100 kcal and about 4.5 g/100 kcal for the infant formula (IF) and the protein levels may be between about 3 g/100 kcal and about 5.5 g/100 kcal for the follow on formula (FOF). In one embodiment, the protein level is about 2.0 g/100 kcal for the IF. The formula comprises lactose or glucose polymers, in addition to the lactose contributed from the dairy, to meet the IFA/Codex minimum to maximum requirements for carbohydrate energy. In one embodiment, the carbohydrate levels are between about 9.0 g/100 kcal and about 14.0 g/100 kcal for IF. In one embodiment, the carbohydrate level is about 10.95 for IF.

In one embodiment, formula comprises goat milk whey and includes a ratio of whey:casein at about 60:40 whey: casein. In one embodiment, the formula comprises bovine whey protein or partially hydrolyzed whey protein.

The formula may optionally include salts. Salts may include counterions selected from chloride, bromide, iodide, and the like. Further salts may include, but are not limited to, fluoride, formate, acetate, propionate, butyrate, glutamate, aspartate, ascorbate, benzoate, carbonate, citrate, carbamate, gluconate, lactate, methyl bromide, methyl sulfate, nitrate, phosphate, diphosphate, succinate, tartrate, malate, citrate, sulfonate, trifluoromethanesulfonate, trichloromethanesulfonate, tribromomethanesulfonate, and trifluoroacetate.

The formula may optionally include prebiotics or probiotics. The formula may also include Vitamin, minerals, and optionally, (Docosahexaenoic acid (DHA)/arachidonic acid (AA), Nucleotides, and other beneficial substances.

### I. Enriched Polyphenol Content and Antioxidant Function

In certain embodiments, the formula of the present invention contains a lipid system engineered to retain significantly higher intrinsic antioxidant content, including phenolic compounds, relative to conventional formulas formulated with fully refined seed oils. Phenolic compounds are plant-derived antioxidants capable of quenching reactive oxygen species (ROS), reducing lipid peroxidation, and protecting mitochondrial membranes during periods of elevated metabolic demand.

Extra-virgin olive oil has been reported to contain between about 100 and about 300 mg/kg total phenolics, while cold-pressed flaxseed oil contains about 10 mg/kg total phenolics. In contrast, fully refined high-oleic seed oils, refined coconut or palm oils, and refined soy or canola oils typically contain ≤2-30 mg/kg total phenolics or non-detectable levels following refining.

When normalized to about 5.5 g fat per 100 kcal, the formula provides approximately between about 0.4 and about 1.2 mg phenolic compounds per 100 kcal, compared to about 0.04 mg per 100 kcal for representative conventional formulas representing an unexpected ≥10-fold to ≥20-fold increase in intrinsic antioxidant delivery.

### II. Oleic-Dominant, Low-PUFA Stress Lipid Architecture

The lipid system of the present invention is further engineered to be oleic-acid dominant and low in polyunsaturated fatty acid (PUFA) stress. This reduces oxidative susceptibility while maintaining essential fatty acid sufficiency.

Inclusion of ruminant-derived fat introduces non-random triacylglycerol (TAG) structures that more closely resemble mammalian milk fat, altering digestion kinetics, micelle formation, and postprandial lipid handling in a manner not achievable with seed-oil-only systems.

Unexpectedly, the combination of oleic dominance, reduced PUFA burden, preserved phytochemicals, and mammalian-inspired TAG architecture produces synergistic improvements in oxidative stability, mitochondrial protection, and metabolic resilience.

### III. Non-Obviousness and Unexpected Results

The present invention is non-obvious over conventional infant formula compositions, which emphasize fatty acid minimum compliance without regard to oxidative pressure, metabolic health, and phytochemical preservation. Prior art teaches away from high-olive-oil systems due to concerns regarding meeting essential fatty acid requirements.

Contrary to such teachings, the disclosed formulations achieve compliance while delivering unexpected oxidative robustness and antioxidant density which better support mitochondrial and metabolic health.

### Regulatory-Compliant Embodiment Clarification

While unrefined or virgin oils may represent a phytochemical upper bound useful for comparative and teaching purposes, the inventive refined-oil embodiments achieve meaningful oxidative and metabolic benefits through lipid architecture of oleic-acid dominance, reduced polyunsaturated fatty acid stress, and optimized linoleic-to-alpha-linolenic acid ratios alone, even in the absence of high intrinsic phytochemical content.

In one embodiment, an infant nutrition composition comprises a refined lipid system delivering between about 3.0 and about 6.0 g total fat per 100 kcal, wherein the lipid system is oleic-acid dominant, exhibits reduced polyunsaturated fatty acid stress, maintains a linoleic acid to alpha-linolenic acid ratio between about 5:1 and about 7:1, and provides enhanced oxidative stability relative to infant formulas predominantly containing higher PUFA seed oils.

In one embodiment, the lipid system further comprises virgin olive and/or avocado oil to introduce higher intrinsic antioxidant levels to further protect metabolic health relative to infant formulas predominantly containing higher PUFA seed oils. In one embodiment, the lipid system provides reduced peroxide value, anisidine value, or Totox value over storage relative to seed-oil-based infant formulas. In one embodiment, the comprises olive oil and/or avocado oil providing greater than about 30%, ideally greater than 60%, of total fat to meet essential linoleic acid requirements. In one embodiment, the lipid system further comprises flaxseed oil, chia oil, walnut oil, or hemp oil at less than about 10%, ideally less than 4%, of total fat to meet essential alpha-linolenic acid requirements. In one embodiment, the lipid system containing virgin olive and/or avocado oil at greater than about 30% of total fat, ideally greater than 60%, to provide levels of intrinsic antioxidants exceeding those of formulas using industrial seed oils.

### Human Milk Comparator and Functional Alignment

Human milk fat is characterized by oleic-acid dominance, moderated linoleic acid content, structured triacylglycerols, and biological antioxidant systems that collectively protect infant mitochondrial function during periods of elevated metabolic demand.

The refined lipid systems of the present invention are engineered to approximate key functional attributes of human milk fat, including oleic dominance, reduced oxidative susceptibility, and intrinsic antioxidants, thereby providing a closer functional alignment than conventional seed-oil-based infant formulas.

### Triacylglycerol Effects as Secondary Consequence

Any triacylglycerol structural effects observed in the disclosed formulations arise as a secondary and emergent consequence of fatty-acid composition and ingredient selection, rather than as a primary design objective or manufacturing step.

References herein to approximation of mammalian or human milk fat are intended to denote approximation of certain functional aspects, including oxidative robustness, oleic-acid dominance, and antioxidant capacity, and do not imply replication of complex human milk chemistry and physiology such as fat globule membrane structure, droplet size engineering, or biological antioxidants.

### Additional Embodiments: Saturated Fat and Omega-6 Modulation

In certain embodiments, the oleic-dominant, low-PUFA lipid systems of the present invention further exhibit a reduced saturated fatty acid (SFA) fraction relative to conventional infant formulas. Elevated dietary saturated fat has been associated with reduced metabolic flexibility and altered lipid handling, whereas lipid systems characterized by higher monounsaturated fatty acid (MUFA) content and moderated saturated fat may support improved metabolic responses. Accordingly, reducing saturated fat content while maintaining oleic-acid dominance constitutes an additional functional advantage arising from the disclosed lipid architecture.

In further embodiments, the lipid system provides moderated omega-6 fatty acid exposure, including controlled linoleic acid content and a balanced omega-6 to omega-3 ratio consistent with international infant nutrition standards. In certain embodiments, omega-6 fatty acids comprise not more than about 15% of total fatty acids, and the omega-6 to omega-3 ratio is maintained between about 5:1 and about 7:1, ranges selected to meet essential fatty acid requirements while avoiding excessive omega-6 biochemical pressure. Such moderation further supports oxidative balance and metabolic resilience.

In one embodiment, the lipid system comprises a saturated fatty acid content lower than that of infant formulas formulated with coconut oil and/or palm oil. In one embodiment, the omega-6 fatty acids comprise not more than about 15% of total fatty acids. In one embodiment, the omega-6 to omega-3 fatty acid ratio is maintained between about 5:1 and about 7:1. In one embodiment, the reduced saturated fatty acid content and moderated omega-6 exposure arise as secondary consequences of oleic-acid dominance and reduced polyunsaturated fatty acid pressure. In one embodiment, the total omega-6 fatty acids are reduced relative to conventional seed-oil-based infant formulas, independent of DHA or ARA supplementation.

Inventive distinction described herein does not arise from compliance with regulatory omega-6 to omega-3 ratio requirements, as both the present invention and conventional infant formulas necessarily fall within accepted nutritional ranges. Rather, the inventive distinction arises from substantially lower total polyunsaturated fatty acid (PUFA) content and reduced linoleic acid exposure relative to conventional infant formulas formulated predominantly with industrial seed oils.

Analysis of representative fatty acid profiles demonstrates that seed-oil-based infant formulas typically exhibit materially higher PUFA fractions and omega-6 content, even when nominal omega-6 to omega-3 ratios are similar. In contrast, the oleic-dominant lipid systems of the present invention achieve reduced biochemical PUFA pressure by lowering absolute omega-6 burden while maintaining essential fatty acid sufficiency.

Accordingly, metabolic and oxidative advantages described herein are attributable to reduced total PUFA exposure and oleic-acid dominance rather than regulatory ratio compliance. This distinction is not taught or suggested by prior art formulations that focus primarily on meeting minimum essential fatty acid ratios through seed-oil blending.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary of the invention and are not intended to limit the scope of what the inventors regard as their invention. However, those of skill in the art should, considering the present disclosure, appreciate that many changes can be made in the specific embodiments which are disclosed and still obtain a like or similar result without departing from the spirit and scope of the invention.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Formulations

Formulation scenarios based on these oils. Essential fatty acid profiles are provided.

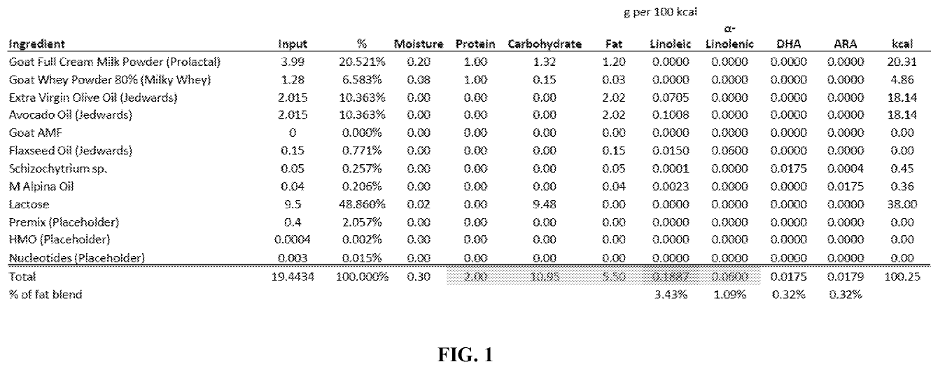

FIG. 1 is a table of a formulation, showing the risk of receiving all oils with fatty acids at the low end of the specification range for linoleic acid. The allowable fatty acid level variability per current specifications is a wide range (Olive oil has a range of 3.5-21% linoleic acid and Avocado has a range of 5-20% linoleic acid).

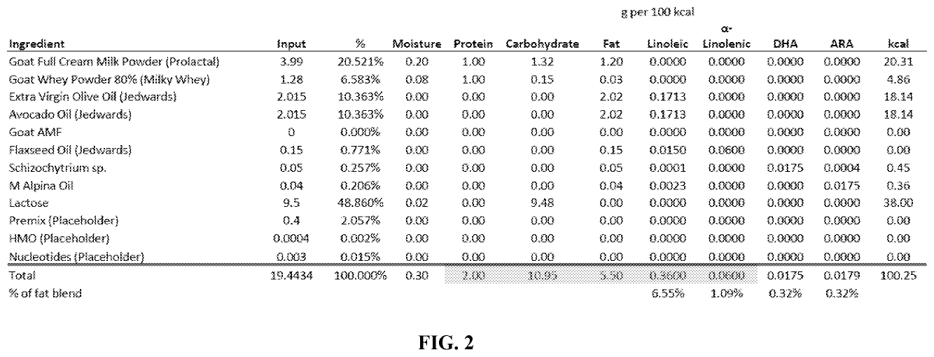

FIG. 2 is a table of a formulation shows the fatty acid profile achievable by increasing the minimum spec value for linoleic acid to 8.5% for both Avocado Oil and Olive oil (from 5% and 3.5%, respectively). This formulation still below the midpoint spec value for both oils.

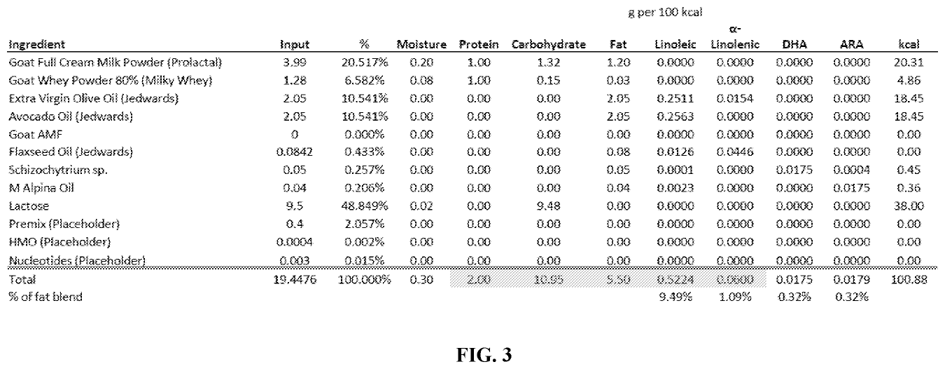

FIG. 3 is a table of a formulation shows the linoleic acid profile achievable by using the mid-point spec value for all oils-linoleic acid is well above the minimum (300 mg/100 kcal). Flaxseed oil usage was able to come down while still maintaining the same 60 mg/100 kcal target for α-linolenic acid used in the previous two formulations—olive oil and Avocado oil were increased accordingly to meet the 5.5 g fat/100 kcal target.

### Example 2: Oxidative Stability Study

### Background and Objective

A seed oil, palm oil free infant formula for the US market is being developed with full cream goat or grass-fed A2 whole milk and a protein composition ideally suitable for different infant age groups (e.g., 0-6 months, 6-12 months). The infant formula is comparing the oil blend to a standard infant formula oil blend from the standpoint of oxidative stability.

### Oil Blends

Oil samples with similar production dates were sourced from Adams oils and were prepared at the EPDI lab
FN518090A is Prototype Blend 1 (sunflower oil, soy and coconut)
FN518090B is Alternative Prototype Blend 2 (sunflower oil, soy, coconut and canola)
FN518090C is the Goldie Blend (avocado oil, coconut and flaxseed)

### Oil Blend Preparation:

Each oil was kept frozen and then thawed out in a water bath set at 100° F. (37.7° C.)

The entire contents of the container were emptied out into a clean, sanitized pail, stirring to make sure all oil fractions were in liquid state and homogeneous.
Individual components were weighed out, to within 0.02 g, and mixed with a 6 cm stir bar at 120 rpm for 10 minutes
A batch of 300 g of each blend was prepared

TABLE 1
Oil Blends

| OIL BLEND | Lot # | % | % | % |
|---|---|---|---|---|
| RBDW Avocado Oil | LOT213099 | | | 74.52 |
| RBDM Exp Org Canola Oil | LOT212151 | | 18.00 | |
| RBDM Exp Org Coconut | LOT212147 | 18.80 | 19.00 | 22.70 |
| Oil | | | | |
| Organic Flaxseed Oil | OFL550111-1 | | | 2.78 |
| RBDM Exp Org Oleic | LOT212150 | 49.50 | 45.00 | |
| Sunflower Oil | | | | |
| RBDM Exp Org Soybean | LOT213000 | 31.70 | 18.00 | |
| Oil | | | | |
| Total | | 100.00 | 100.00 | 100.00 |

### Testing Plan

Submit the three blends for PV analysis as a benchmark—as a check as compared to specification/CoA results.

TABLE 2

| | | Peroxide Value |
|---|---|---|
| | | on COA, |
| OIL TYPE | Lot # | mEq/kg |
| RBDW Avocado Oil | LOT213099 | 0.2 |
| RBDM Exp Org Canola Oil | LOT212151 | 0.02 |
| RBDM Exp Org Coconut Oil | LOT212147 | NA |
| Organic Flaxseed Oil | OFL550111-1 | 6 |
| RBDM Exp Org Oleic Sunflower Oil | LOT212150 | 0.02 |
| RBDM Exp Org Soybean Oil | LOT213000 | 0.4 |

### 2. Submit the Blends for Oxidative Stability Index Testing (Rancimat Method)

Standard method is carried out at 110° C. and air flow at 9 L/h. A second test was carried out at 90° C.; lower temperatures may permit greater differences in OSI between the 3 oil blends.

### Analysis Results

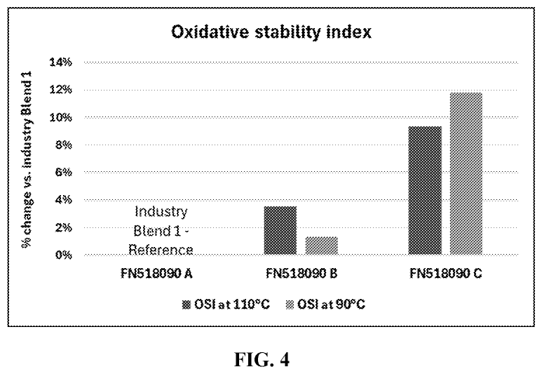

As shown in FIG. 4, the oil blend of the present invention (designated as FN518090 C) had higher comparative oxidative stability at 15.31 OSI (h @ 110 C) and 63.66 OSI (h @ 90 C) than both other blends PV (designated as FN518090 A and FN518090 B), for both methods used (standard 110° C., as well as the 90° C.).

| OSI (h @ 110° C.) | FN518090 A | FN518090 B | FN518090 C |
|---|---|---|---|
| OSI (h @ 110° C.) | 13.88 | 14.39 | 15.31 |
| % change vs. industry Blend 1 | 0% | 4% | 9% |

| OSI (h @ 90° C.) | FN518090 A | FN518090 B | FN518090 C |
|---|---|---|---|
| OSI (h @ 90° C.) | 56.17 | 56.92 | 63.66 |
| % change vs. industry Blend 1 | 0% | 1% | 12% |

When tested at 110° C., the oil blend of the present invention had a stability which was ~1.5 hrs longer than the reference Blend 1, while samples tested at 90° C. had OSI with about 7.5 hrs longer than the reference.

Using the lower method temperature resulted in higher percentage differences in OSI versus the industry reference Blend 1, with about 12% higher OSI than the reference for the 90° C. method.

The lab has run a control oil sample alongside the oil blends using the standard method (at 110° C.), which had a relative standard deviation (RSD) of about 1.9%

Based on technical literature, the equipment used in the lab (Methrohm Rancimat) provides very reproducible results, with a coefficient of variability of less than between about 1.5 and about 2%.

All publications and patent applications mentioned in this specification are herein incorporated by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A nutritional composition comprising: a lipid system including a hierarchical fatty acid architecture and the nutritional composition includes a caloric content and a total fatty acid content, wherein the total fat acids provides approximately between about 20% and about 60% of the caloric content, the total fatty acid content includes a monounsaturated fatty acid concentration and a polyunsaturated fatty acid concentration; the monosaturated fatty acid concentration comprises between about 50% and about 75% of total fatty acid content, the polyunsaturated fatty acid concentration comprising no more than about 15% of the total fatty acid content, the polyunsaturated fatty acid concentration includes linoleic acid comprising between about 6% and about 12% of total fatty acid content, a lauric acid content comprising less than 4% of the total fatty acid content, and the lipid system includes at least one mammalian-derived fat source.

2. The composition of claim 1, wherein the monounsaturated fatty acid concentration comprises between about 58% and about 60% of the total fatty acid content.

3. The composition of claim 2, wherein the polyunsaturated fatty acid concentration comprises between about 8% and about 13% of the total fatty acid content.

4. The composition of claim 3, wherein the mammalian-derived fat source comprises dairy fat.

5. The composition of claim 4, wherein the lipid system includes milk fat globule membrane components and a concentration of saturated fatty acids between about 15% and about 30% of the total fatty acid content.

6. The composition of claim 5, wherein the lipid architecture modulates post-prandial metabolic signaling.

7. The composition of claim 6, wherein the hierarchical fatty acid architecture modulates metabolic signaling of a subject through coordinated fatty acid class distribution and controlled polyunsaturated fatty acid exposure, including an oxidative stability at 15.31 OSI.

8. The composition of claim 7, wherein the hierarchical fatty acid architecture emulates structural characteristics of mammalian milk lipid systems and inclusion of ruminant-derived lipid membrane components.

9. The composition of claim 8, wherein the hierarchical fatty acid architecture is configured to reduce incorporation of oxidatively unstable polyunsaturated fatty acids into biological membranes through constrained dietary PUFA exposure and monounsaturated fatty acid dominance.

10. The composition of claim 9, wherein the composition is formulated as an infant formula.

11. A milk formulation supporting metabolic health and mitochondrial function in an infant, comprising: a formulation including an effective amount of a plant oils, wherein plant oils comprise requisite linoleic acid and α-linolenic fatty acids levels, wherein the total fat energy is between about 3.0 g/100 kcal and about 6.0 g/100 kcal and the alpha-linolenic acid is a minimum of 50 mg/100 kcal.

12. The formulation of claim 11, wherein the linoleic acid comprises about 300 mg and the alpha-linolenic acid is about 60 mg.

13. The formulation of claim 12, wherein the plant oils comprise phytochemicals including bio-actives and antioxidants.

14. The formulation of claim 13, wherein the formulation is an infant or toddler formula and does not include seed oils including Soy, Canola, Sunflower, Safflower, Corn oils.

15. The formulation of claim 14, wherein the infant or toddler formula comprising proteins, carbohydrates, fats, vitamins, minerals, and optionally, DHA/AA, Prebiotics, nucleotides, and other beneficial substances.

16. The formulation of claim 15, wherein the infant or toddler formula comprises a goat milk based formula or a cow-milk based formula.

17. The formulation of claim 16, wherein the plant oils providing the linoleic acid comprise Olive, Avocado, Flax, Chia, Walnut, or Hemp oil alone or in any ratio.

18. The formulation of claim 11, wherein a ratio of linoleic:a linolenic is between about 5:1 and about 7:1.

19. The formulation of claim 16, wherein the plant oil comprises olive oil or avocado oil greater than about 30% of the fat in the formula.

20. The formulation of claim 17, wherein the plant oils providing the alpha-linolenic acid comprise flax, chia, walnut or hemp oil alone or in any ratio and not exceeding 10% of the fat in the formula.

21. The formulation of claim 11, wherein the lipid system comprises a total polyunsaturated fatty acid content lower than that of infant formulas formulated predominantly with industrial seed oils.

22. The formulation of claim 11, wherein the lipid system comprises a linoleic acid content materially lower than that of seed-oil-based infant formulas while maintaining essential fatty acid sufficiency.

23. The formulation of claim 11, wherein metabolic and oxidative benefits arise from reduced absolute omega-6 fatty acid exposure rather than from omega-6 to omega-3 ratio manipulation.

24. The formulation of claim 11, wherein total omega-6 fatty acids are reduced relative to conventional seed-oil-based infant formulas, independent of DHA or ARA supplementation.

25. A method of promoting metabolic health and mitochondrial function in an infant, comprising:
a. providing a milk formulation comprising an effective amount of plant oils, wherein plant oils comprise requisite linoleic acid and α-linolenic fatty acids, wherein the total fat energy is between about 3.0 g/100 kcal and about 6.0 g/100 kcal and the alpha-linolenic acid is a minimum of 50 mg/100 kcal; and
b. administering an effective dose of the plant oils to an infant or a toddler.

26. The method of claim 25, further comprising reducing obesity rates in infants or toddlers.
